(19) United States
(12) Plant Patent Application Publication
(10) Pub. No.: US 2007/0271654 P1
(43) Pub. Date: Nov. 22, 2007

(54) IMPATIENS PLANT NAMED 'IMDOREDTWO'

(75) Inventor: Monica Maria Adelheid Sanders, Grootebroek (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/438,610

(22) Filed: May 22, 2006

(51) Int. Cl. A01H 5/00

(52) U.S. Cl. PLT/317

(57) ABSTRACT

A new Impatiens plant, characterised particularly as to novelty by a compact habit, freely branching with large, double, red flowers, flowering above the foilage.

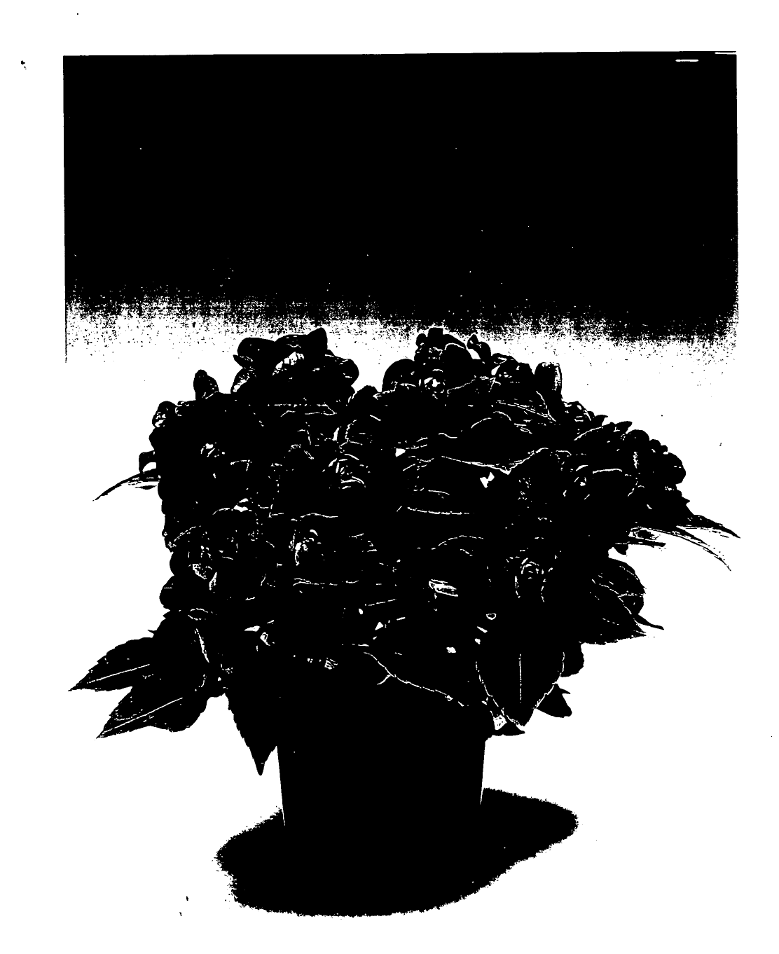

### LATIN NAME OF THE GENUS AND SPECIES OF THE PLANT CLAIMED

Impatiens walleriana

### VARIETAL DENOMINATION

‘Imdoredtwo’

### BACKGROUND OF THE NEW PLANT

The present invention comprises a new distinct cultivar of Impatiens, botanically known as Impatiens walleriana.

The new Impatiens is a product of a planned breeding program conducted in Enkhuizen, Netherlands. The new Impatiens cultivar is compact growing, early flowering, floriferous with large, double flowers, flowering above the foliage and with attractive foilage.

The present cultivar originated from a pollination made in 2001, of a proprietary Impatiens selection identified as ‘S 345-2,’ not patented, as the female, or seed parent, with a proprietary Impatiens selection identified as ‘S 345-1,’ not patented, as the male, or pollen parent.

The present cultivar has been repeatedly asexually reproduced by cuttings in Enkhuizen, Netherlands, in Angers, France, in Gilroy, USA over a four year period. The distinctive characters of this new Impatiens are stable and reproduced true to type in successive generations of asexual reproduction. It takes 7 to 9 weeks to produce a finished plant, depending on the temperature.

This new Impatiens plant is an annual in most climatical zones in the US, only in zones 9 and 10 it is a perennial plant.

### DESCRIPTION OF THE DRAWING

This new Impatiens plant is illustrated by the accompanying photographic drawing which shows blooms, buds and foliage of the plant in full color, the color showing being as true as can be reasonably obtained by conventional photographic procedures.

### DESCRIPTION OF THE NEW CULTIVAR

The following detailed descriptions set forth the distinctive characteristics of this new Impatiens. The data which defines these characteristics were collected from asexual reproductions carried out in Enkhuizen, Netherlands. The plant history was taken on 25 week old plants, blossomed under natural light in the field.

Color readings were taken in the office under ambient light. Color references are primarily to the R.H.S. Color Chart of The Royal Horticultural Society of London.

Plants of the new Impatiens differ primarily from the plants of the female parent selection in the following characteristics:

Plants of the new Impatiens have red double flowers and flowers only have petals, whereas the plants of the female have red flowers and flowers have a pollen cap and stigma.

Plants of the new Impatiens differ primarily from the male plant selection in the following characteristics:

Plants of the new Impatiens have red double flowers and flowers only have petals, whereas the plants of the male have red flowers and flowers have a pollen cap and stigma.

TABLE 1
DIFFERENCES BETWEEN THE NEW CULTIVAR ‘IMDOREDTWO’
AND A SIMILAR CULTIVAR

| | | ‘Balolecher’ |
|---|---|---|
| | | (U.S. Plant |
| | | Patent No. |
| | ‘Imdoredtwo’ | 13,668) |
| Flower color | Red | Dark red |
| Leaf color | Dark green | Medium green |
| Flower size | Large | Smaller |

PARENTAGE

| | |
|---|---|
| Female parent: | ‘S 345-2,’ not patented |
| Male parent: | ‘S 345-1,’ not patented |

PROPAGATION

| | |
|---|---|
| Type cutting: | Terminal cuttings |
| Time to initiate roots: | 10-12 days at 20 degrees C. |
| Time to produce a rooted cutting: | 4 weeks |
| Root description: | Fine, fibrous and white in color |

PLANT DESCRIPTION

| | |
|---|---|
| General appearance: | Compact, mounded and outwardly |
| | spreading. Appropriate for 12 cm |
| | pots and large containers |
| Growth and branching habit: | Compact growing and early |
| | flowering, well branched |
| Crop time: | 7-9 weeks, starting from an |
| | unrooted cutting in a 10.5 cm pot |
| Plant height: | 15 cm |
| Plant diameter or spread: | 49 cm |

LATERAL BRANCHES

| | |
|---|---|
| Length: | About 20.5 cm |
| Diameter: | About 6 mm |
| Internode length: | About 2.5 cm |
| Texture: | Smooth |
| Color: | 146C |
| Anthocyanin may be present: | 59B |

FOLIAGE DESCRIPTION

| | |
|---|---|
| Arrangement: | Simple, alternate |
| Length: | About 4.5 cm |
| Width: | About 2.5 cm |
| Shape: | Ovate |
| Apex: | Acuminate |
| Base: | Attenuate |
| Margin: | Serrate, rounded |
| Texture: | Glabrous, smooth |
| Venation pattern: | Pinnate, arcuate |
| Color, developing foliage, | 137A |
| upper surface: | |
| Color, fully expanded foilage, | 137C |
| lower surface: | |
| Color, developing and fully | 138B |
| expanded foliage, lower surface: | |
| Venation, upper and lower | 138D |
| surfaces: | |
| Petiole, length: | About 1.2 cm |
| Petiole, diameter: | About 2 mm |
| Color: | 49C with green |

FLOWER DESCRIPTION

| | |
|---|---|
| Flower type and flowering habit: | Numerous and consistently large |
| | fully double flowers. Freely and |
| | continuously flowering. Flower |
| | buds open similar to a rose in |
| | fullness; flowers rounded. Flowers |
| | arise from leaf axils. Typically |
| | about 35 flowers and flower buds |
| | per lateral branch. Flowers |
| | positioned above and beyond the |
| | foliage and typically face mostly |
| | outward. Flowers last about five to |
| | seven days under greenhouse |
| | conditions. Flowers not persistent. |
| | Flowers not fragrant. |
| Flowering season: | Year round under greenhouse |
| | conditions. In the garden, flowering |
| | is continuous from Spring until Fall. |

Flower size

| | |
|---|---|
| Diameter: | About 4 cm |
| Depth: | About 2 cm |

FLOWER BUDS

| | |
|---|---|
| Length, at stage for showing | About 1.1 cm |
| color: | |
| Diameter, at stage for showing | About 9 mm |
| color: | |
| Shape: | Ovoid |
| Color: | 58C |

PETALS

| | |
|---|---|
| Quantity/arrangement: | About 43 per flower; imbricate |
| Length, outer petals: | About 2.5 cm |
| Width, outer petals: | About 2 cm |
| Shape: | Oval to obovate |
| Apex: | Emarginate to retuse |
| Base: | Shortly attenuate |
| Margin: | Entire |
| Texture, upper and lower surfaces: | Velvety, smooth |

Color

| | |
|---|---|
| When opening, upper surface: | 45B |
| When opening, lower surface: | 46D |
| Fully opened, upper surface: | 46C to 46B |
| Fully opened, lower surface: | 44D |

SPURS

| | |
|---|---|
| Length: | About 2.2 cm |
| Color: | 49C |

SEPALS

| | |
|---|---|
| Quantity/arrangement: | Five in a single whorl |
| Calyx length: | About 3 mm |
| Calyx diameter: | About 1 mm |
| Shape: | Elliptic |
| Apex: | Acute |
| Margin: | Entire |
| Texture, upper and lower surfaces: | Smooth |

Color

| | |
|---|---|
| Upper surface: | 146C |
| Lower surface: | 144B |

PEDUNCLES

| | |
|---|---|
| Length: | About 2.3 cm |
| Diameter: | About 2 mm |
| Angle: | About 45 degrees to the lateral |
| | branch |
| Strength: | Moderately strong |
| Color: | 152C |

Reproductive organs: None observed
Seed/fruit development: Seed and fruit development has not been observed

What is claimed is:

1. A new and distinct cultivar of Impatiens plant named ‘Imdoredtwo,’ as substantially illustrated and described herein.
